(19) United States
(12) Plant Patent Application Publication
(10) Pub. No.: US 2019/0239402 P1
(43) Pub. Date: Aug. 1, 2019

(54) Rosa L. variety named '80103WABARA'

(50) ;

(71) Applicant: Rose Universe Co., Ltd., Shiga (JP)

(72) Inventor: Keiji Kunieda, Shiga (JP)

(21) Appl. No.: 15/932,569

(22) Filed: Mar. 15, 2018

(30) Foreign Application Priority Data
Jan. 26, 2018 (JP) PVP 32809

(51) Int. Cl. A01H 6/74

(52) U.S. Cl. PLT/103

(57) ABSTRACT

Described herein is a new and distinct rose variety named ‘80103WABARA’.

### CROSS-REFERENCES TO RELATED APPLICATIONS

This application claims priority to Japanese Plant Variety Protection Application No. 32809, filed Jan. 26, 2018.

Genus and species: The rose variety of this invention is botanically identified as Rosa L.

Variety denomination: The variety denomination is ‘80103WABARA’.

### BACKGROUND OF THE INVENTION

The present invention is a new and distinct variety of a Rosa L. rose plant obtained from a cross between maternal variety ‘An’ and an unnamed paternal variety of unknown parentage owned by the breeder. The cross was performed by hand pollination in May 2014 and seeds were planted in January 2015. ‘80103WABARA’ was selected from the varieties that flowered in September 2017. Stable characteristics were confirmed following selection.

### BRIEF SUMMARY OF THE INVENTION

‘80103WABARA’ is reproduced by grafting. ‘80103WABARA’ can be distinguished from the maternal variety, ‘An’, by: (1) a large number of petals (128 petals compared to 54 for ‘An’); (2) color grouping (white compared to pink blend for ‘An’); (3) main color of the inner side of the petal (RHS 158D compared to RHS 49C for ‘An’); and (4) petal secondary color (RHS 4D compared to absent for ‘An’). ‘80103WABARA’ is also distinguished from comparison variety ‘Rikuhotaru’ by the main color of the inner side of the petal (RHS 158D compared to RHS 13D for ‘Rikuhotaru’).

The variety ‘80103WABARA’ is maintained at Shiga-ken Moryama-shi, 1465 Sugie-cho 524-0062, Japan and is maintained in soil cultivation. The variety is primarily used as a potted plant and for cut flowers. Planting in Japan is from mid-October to late December. Grafting is from late September to early October. The plant will flower annually when grown in a greenhouse.

### BRIEF DESCRIPTION OF THE PHOTOGRAPHS

The accompanying photographs illustrate the appearance of the new Rosa L. variety. The colors in the photographs are depicted as nearly true as is reasonably possible to obtain in colored reproductions of this type. Colors in the photographs may differ slightly from the color values cited in the detailed botanical description.

FIG. 1 shows a branch with a flower and leaves.

FIG. 2 shows the prickles on a branch.

FIG. 3 shows leaves with leaflets.

FIG. 4 shows a close-up side view of a flower.

FIG. 5 shows a close-up front view of a flower.

FIG. 6 shows sepals at the base of a flower.

FIG. 7 shows a close-up view of sepals at the base of a flower.

FIG. 8 shows individual petals.

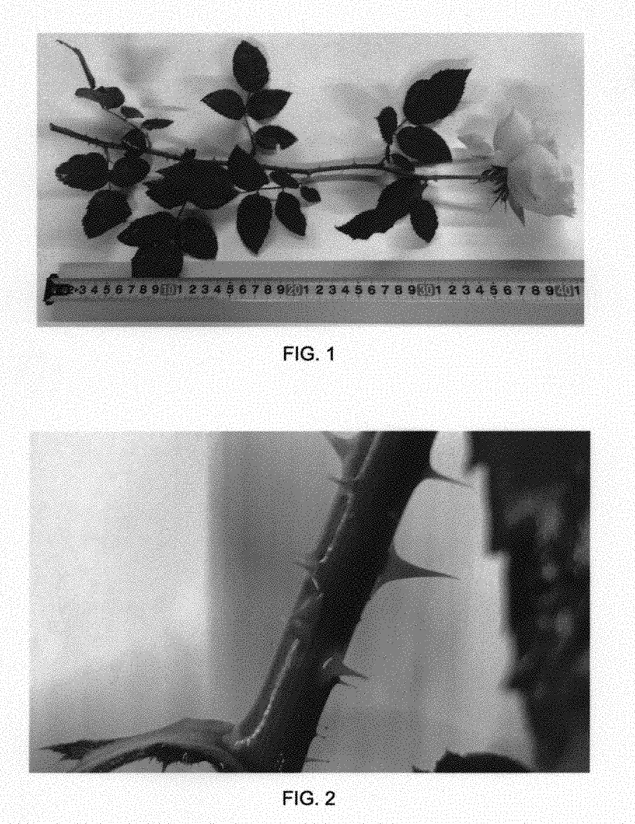

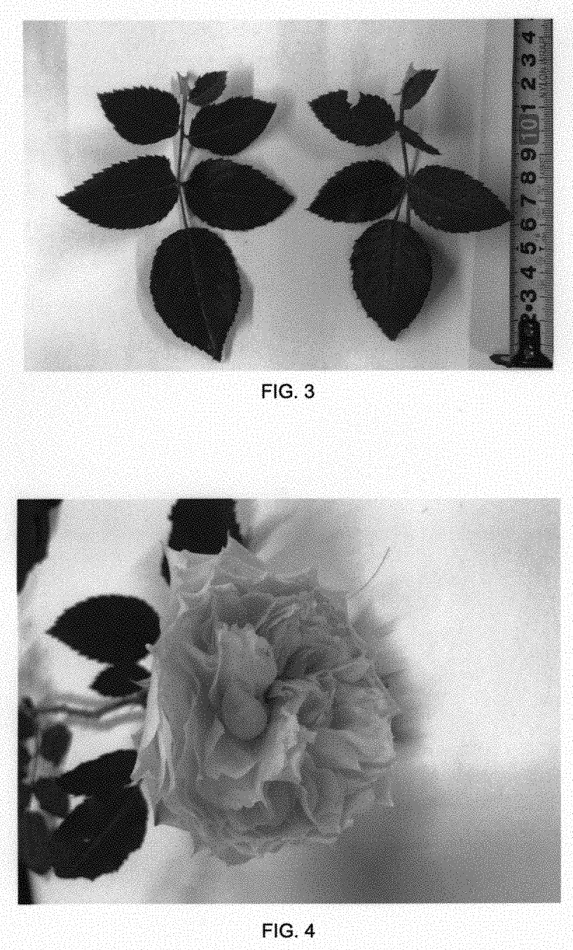

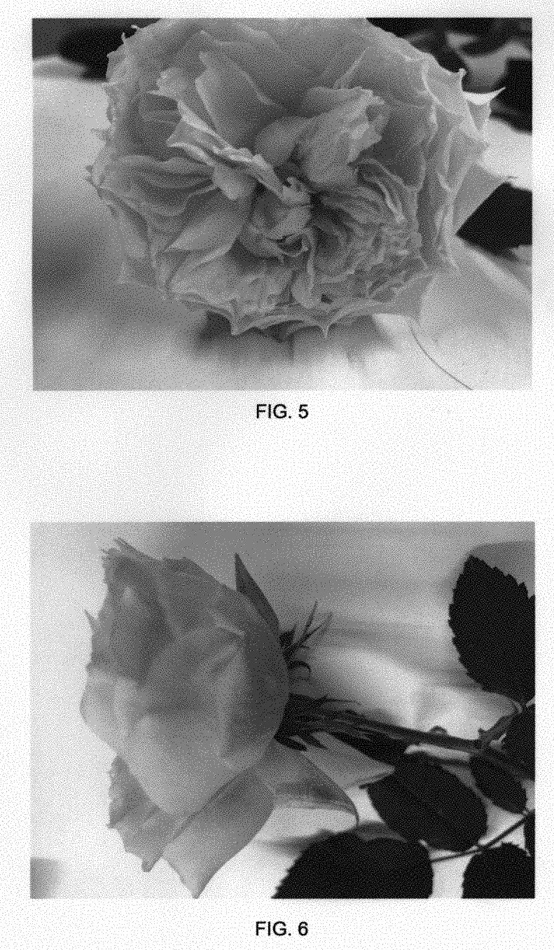

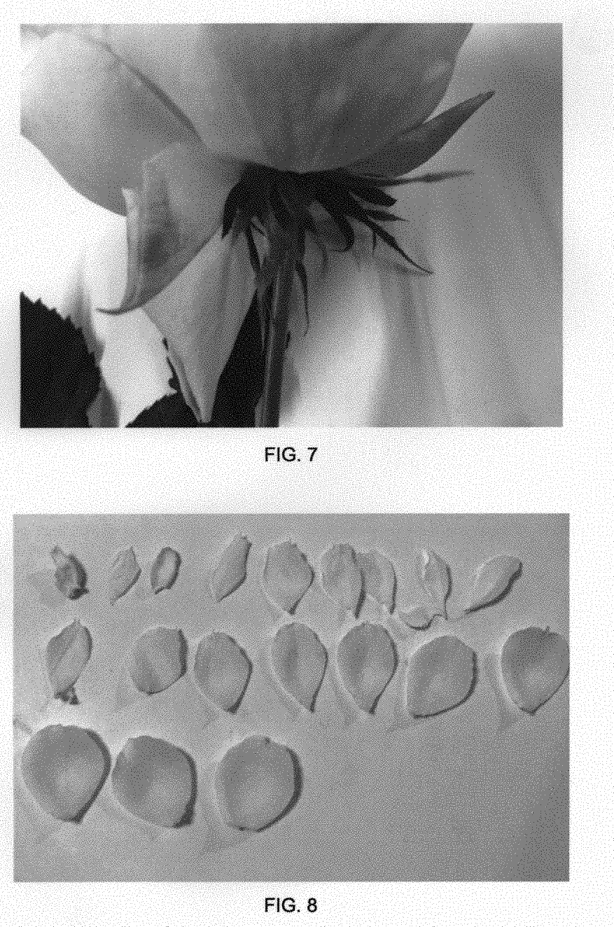

### DETAILED BOTANICAL DESCRIPTION

The following is a detailed description of ‘80103WABARA’ with color descriptions based on The Royal Horticultural Society (R.H.S.) Colour Chart for those colors referred to by R.H.S. designation.
Characteristics of variety: Grade value numbers shown in the last column are with reference to the International Union for Plant Varieties (UPOV) and Japanese Tables of Characteristics for Rosa L.

| | | | Applicable Variety |
|---|---|---|---|
| Characteristic | Name of | | Grade Value |
| No. | Characteristic | Characteristic | (Characteristic Value) |
| 1 | Plant: growth type | shrub | 4 |
| 2 | Plant: growth habit | semi upright | 3 |
| 3 | Plant: height | medium | 5 (39.5 cm) |
| 4 | Young shoot: anthocyanin | absent | 1 |
| | coloration | | |
| 6 | Stem: number of prickles | many | 9 |
| 7 | Prickles: predominant color | reddish | 3 |
| 8 | Leaf: size | large | 7 (13 cm) |
| 9 | Leaf: intensity of green color | medium | 5 |
| 10 | Leaf: anthocyanin coloration | absent | 1 |
| 11 | Leaf: glossiness of upper side | weak | 3 |
| 12 | Leaflet: undulation of margin | very weak | 1 |
| 13 | Terminal leaflet: shape of blade | medium | 2 |
| | | elliptic | |
| 14 | Terminal leaflet: shape of base | rounded | 3 |
| | of blade | | |
| 15 | Terminal leaflet: shape of apex | acuminate | 1 |
| | of blade | | |
| 16 | Flowering shoot: flowering | present | 9 |
| | laterals | | |
| 17 | Flowering shoot: number of | very few | 1 (1 lateral) |
| | flowering laterals | | |
| 18 | Flowering shoot: number of | very few | 1 (1 flower) |
| | flowers | | |
| 19 | Flowering shoot: number of | very few | 1 (1 flower) |
| | flowers per lateral | | |
| 21 | Flower: type | double | 3 |
| 22 | Flower: number of petals | very many | 9 (128 petals) |
| 23 | Flower: color group | white | 1 |
| 25 | Flower: density of petals | dense | 7 |
| 26 | Flower: diameter | large | 7 (9 cm) |
| 27 | Flower: shape | star-shaped | 3 |
| 28 | Flower: profile of upper part | flat | 1 |
| 29 | Flower: profile of lower part | flat | 2 |
| 30 | Flower: fragrance | strong | 3 |
| 31 | Sepal: extensions | very strong | 9 |
| 32 | Petal: reflexing of petals | absent | 1 |
| | one-by-one | | |
| 33 | Petal: shape | elliptic | 1 |
| 34 | Petal: incisions | weak | 3 |
| 15 | Petal: reflexing of margin | very strong | 9 |
| 36 | Petal: undulation | very strong | 9 |
| 37 | Petal: size | medium | 5 |
| 38 | Petal: length | long | 7 (4 cm) |
| 39 | Petal: width | broad | 7 (4 cm) |
| 40 | Petal: number of colors on inner | two | 2 |
| | side | | |
| 42 | Petal: main color on inner side | RHS | 158B |
| 43 | Petal: secondary color | RHS | 4D |
| 45 | Petal: distribution of secondary | stripes | at base |
| | color on inner side | | |
| 47 | Petal: basal spot on the inner | present | 9 |
| | side | | |
| 48 | Petal: size of basal spot on inner | very small | 1 |
| | side | | |
| 49 | Petal: color of basal spot on | medium | 4 |
| | inner side | yellow | |
| 50 | Petal: main color on the outer | RHS | 67A |
| | side | | |
| 51 | Outer stamen: predominant color | light yellow | 3 |
| | of filament | | |

What is claimed is:

1. A new and distinct variety of a Rosa L. rose plant having the characteristics substantially as described and illustrated herein.
